(19) United States
(12) Patent Application Publication
(10) Pub. No.: US 2003/0187064 A1
(43) Pub. Date: Oct. 2, 2003

(54) Use

(76) Inventors: Aksel Buchter-Larsen, Charlottenlund (DK); Andrew John Morgan, Vedbaek (DK); Shukun Yu, Malmoe (SE)

Correspondence Address: FROMMER LAWRENCE & HAUG, 745 FIFTH AVENUE- 10TH FL., NEW YORK, NY, 10151, US

(21) Appl. No.: 10/283,936

(22) Filed: Oct. 30, 2002

Related U.S. Application Data
(60) Provisional application No. 60/343,368, filed on Dec. 21, 2001.

(30) Foreign Application Priority Data
Oct. 31, 2001 (GB) 0126186.6

(51) Int. Cl. A61K 31/366; A61K 31/365

(52) U.S. Cl. 514/460; 514/473

(57) ABSTRACT

The present invention provides use in medicine of a cyclic compound having Formula I,

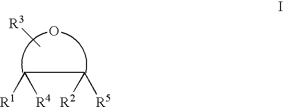

or a derivative thereof,

wherein R¹ and R² are independently selected from —OH, ═O, and OR′, wherein R′ is H or —COR″, and R″ is C₁₋₁₀alkyl;

wherein R³ is a substituent comprising an —OH group;

wherein R⁴ and R⁵ are each independently selected from a hydrocarbyl group, H, OH or ═O, or represent a bond with an adjacent atom on the ring of the cyclic compound.

The invention further relates to an antimicrobial for use against Bacillus anthracis of a cyclic compound of Formula I.

### CROSS REFERENCE TO RELATED APPLICATIONS

This application claims priority to provisional application serial No. 60/343,368, filed Dec. 21, 2001, entitled “Use,” and U.K. application no. 0126186.6 filed Oct. 31, 2001; both of which are incorporated herein by reference, together with any documents therein cited and any documents cited or referenced in therein cited documents. Reference is made to U.S. Provisional Patent Applications Serial Nos.: 60/343,313, filed Dec. 21, 2001, entitled “Ascopyrone P Synthase”; 60/343,485, filed Dec. 21, 2001, entitled “Sequences”; 60/343,447, filed Dec. 21, 2001, entitled “1,5-Anhydro-D-Fructose Dehydratase”; and 60/343,316, filed Dec. 21, 2001, entitled “Process” incorporated herein by reference, together with any documents therein cited and any documents cited or referenced in therein cited documents. Reference is also made to the U.S. Utility patent applications based on the four referenced U.S. Provisional Patent Applications which are filed concurrently herewith as Attorney reference Nos.: 674509-2040.1, 674509-2041.1, 674509-2042.1 and 674509-2043.1. All documents cited herein and all documents cited or referenced in herein cited documents are hereby incorporated herein by reference.

### SUMMARY OF THE INVENTION

The present invention relates to antimicrobial agents. More specifically, the invention relates to the antimicrobial activity of a series of anhydrofructose derivatives.

Bacillus anthracis is gram positive, aerobic, spore forming bacillus that is a major cause of disease in man and mammals. Numerous other species within the Bacillus genus are widely distributed in nature and are commonly found in soil, water and dust samples.

Historically, considerable attention has focused on the genus Bacillus because of the significance of anthrax, the disease caused by Bacillus anthracis. Due to its ability to produce spores, Bacillus anthracis is extremely resistant to adverse chemical and physical environments.

Anthrax is primarily a disease of herbivorous animals but the disease may be contracted by humans that accidentally encounter this disease in an agricultural setting, usually with the development of a local skin infection that may become generalised. The disease may also be contracted in an industrial setting during the processing of hides or animal hair with the resultant inhalation of anthrax and the production of a virulant type of pneumonia. In rare cases, the disease may also be acquired by ingestion.

The present invention seeks to provide antimicrobial agents that are active against a range of microorganisms, and more specifically, against Bacillus anthracis. In particular, the invention seeks to provide antimicrobial agents that are useful in medicine.

The present invention relates to antimicrobial agents suitable for use in food/feedstuffs and in non-food applications.

Food degradation from various sources is recognised in the literature and individual chemicals are known which will inhibit one aspect or another of degradation derived from a single source. Degradation, and the loss of colour or flavour of freshly cut plant parts are known to be caused by oxidation, enzymes, microbes, and metal ions. For example, acidulants are known to prevent microbial degradation by maintaining a relatively low pH environment but their effectiveness is only temporary.

To date, however, the use of chemical substances has been severely limited because on the one hand they have to be safe from a toxicological view point, but on the other hand they must not influence the product sensorically.

Thus, a further aspect of the invention seeks to alleviate the problems associated with prior art chemical substances and to provide new antimicrobial compositions based on anhydrofructose derivatives. More specifically, the invention seeks to provide antimicrobial agents that are suitable for use in both foodstuffs/feed and in non-food applications.

In a first aspect, the invention provides the use in medicine of a cyclic compound having Formula I,

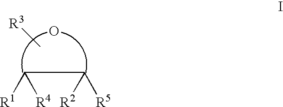

or a derivative thereof,

wherein R¹ and R² are independently selected from —OH, ═O, and OR′, wherein R′ is H or —COR″, and R″ is C₁₋₁₀alkyl;

wherein R³ is a substituent comprising an —OH group;

wherein R⁴ and R⁵ are each independently selected from a hydrocarbyl group, H, OH or ═O, or represent a bond with an adjacent atom on the ring of the cyclic compound.

Additionally, the invention provides a method of treatment comprising administering the above cyclic compound.

A second aspect of the invention relates to the use of a compound having Formula I, or a derivative thereof,

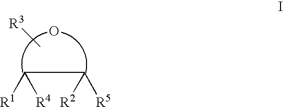

or a derivative thereof,

wherein R¹ and R² are independently selected from —OH, ═O, and OR′, wherein R′ is H or —COR″, and R″ is C₁₋₁₀alkyl;

wherein R³ is a substituent comprising an —OH group;

wherein R⁴ and R⁵ are each independently selected from a hydrocarbyl group, H, OH or ═O, or represent a bond with an adjacent atom on the ring of the cyclic compound;

in the preparation of a medicament for the treatment of a condition associated with the presence of one or more microrganisms.

As used herein, the term “treatment” includes curative effects, alleviation effects, and prophylactic effects.

By way of example, the medicament may be used to treat a condition associated with the presence of one or more microrganisms by preventing and/or inhibiting the growth of, and/or killing the microoganisms.

As used herein, the term “preparation of a medicament” includes the use of a compound of formula I directly as the medicament in addition to its use in a screening programme for further antimicrobial agents or in any stage of the preparation of such a medicament.

Preferably, the microorganism is selected from Listeria, Salmonella, Bacillus, Saccharomyces, Pseudomonas, Clostridium, Lactobacillus, Brochothrix, Micrococcus, Yersinia, Enterobacter, Escherichia, Zygosaccharomyces and Staphylococcus.

Even more preferably, the microorganism is selected from Listeria monocytogenes, Listeria innocua, Salmonella Typhimurium, Salmonella sp., Bacillus cereus, Bacillus subtilis, Saccharomyces cerevisiae, Saccharomyces cerevisiae var. paradoxus, Saccharomyces carlsbergensis, Pseudomonas fluorescens, Clostridium sporogenes, Lactobacillus sake, Brochothrix thermosphacta, Micrococcus luteus, Yersinia enterocolitica, Enterobacter aerogenes, E. coli, Staphylococcus aureus, Bacillus anthracis and Zygosaccharomyces bailii.

In a particularly preferred embodiment, the microorganism is Bacillus anthracis.

In a third aspect, the invention provides an antimicrobial composition or compound for use against Bacillus anthracis, said compound is or said composition comprises a cyclic compound having Formula I,

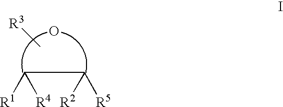

or a derivative thereof,

wherein R¹ and R² are independently selected from —OH, ═O, and OR′, wherein R′ is H or —COR″, and R″ is C₁₋₁₀alkyl;

wherein R³ is a substituent comprising an —OH group;

wherein R⁴ and R⁵ are each independently selected from a hydrocarbyl group, H, OH or ═O, or represent a bond with an adjacent atom on the ring of the cyclic compound.

In a fourth aspect, the invention relates to a process for preventing and/or inhibiting the growth of, and/or killing Bacillus anthracis in a material, the process comprising the step of contacting the material with a cyclic compound having Formula I,

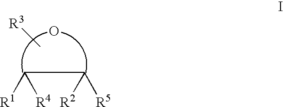

or a derivative thereof,

wherein R¹ and R² are independently selected from —OH, ═O, and OR′, wherein R′ is H or —COR″, and R″ is C₁₋₁₀alkyl;

wherein R³ is a substituent comprising an —OH group;

wherein R⁴ and R⁵ are each independently selected from a hydrocarbyl group, H, OH or ═O, or represent a bond with an adjacent atom on the ring of the cyclic compound.

In a fifth aspect, the invention relates to the use of a compound having Formula I, or a derivative thereof,

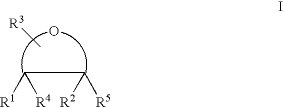

or a derivative thereof,

wherein R¹ and R² are independently selected from —OH, ═O, and OR′, wherein R′ is H or —COR″, and R″ is C₁₋₁₀alkyl;

wherein R³ is a substituent comprising an —OH group;

wherein R⁴ and R⁵ are each independently selected from a hydrocarbyl group, H, OH or ═O, or represent a bond with an adjacent atom on the ring of the cyclic compound;

for preventing and/or inhibiting the growth of, and/or killing Bacillus anthracis in a material.

In respect of said fourth and fifth aspects, the material may be a foodstuff or feed. Thus, in a preferred aspect, the present invention relates to antimicrobial substances that are suitable for use in foodstuffs and/or feed to inhibit food poisoning and spoiling bacteria contained therein.

In another preferred embodiment of said fourth and fifth aspects, the material is a non-food material.

Thus, the present invention also relates to antimicrobial substances for use in non-food applications such as surface cleaning, cleaning of fabrics (laundry), and in cosmetic and/or pharmaceutical products.

Thus in a further aspect, the invention relates to a process for preventing and/or inhibiting the growth of, and/or killing a mico-organism in a non-food material, the process comprising the step of contacting the material with a cyclic compound having Formula I,

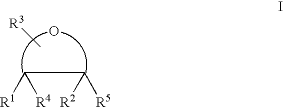

or a derivative thereof,

wherein R¹ and R² are independently selected from —OH, ═O, and OR′, wherein R′ is H or —COR″, and R″ is C₁₋₁₀alkyl;

wherein R³ is a substituent comprising an —OH group;

wherein R⁴ and R⁵ are each independently selected from a hydrocarbyl group, H, OH or ═O, or represent a bond with an adjacent atom on the ring of the cyclic compound.

The compounds of the invention are particularly advantageous in such non-food applications due to their biodegradability and instablility. Moreover, since many of the compounds of formula I may be derived from starch, they are ideally suited for cosmetic, medical and surface cleaning purposes in view of their low toxicity. We have also found that the compounds of the present invention may be active against caries (tooth decay).

By way of definition, the term “antimicrobial” refers to a substance that kills or prevents or inhibits the growth or reproduction of micro-organisms. Antimicrobials are generally classified according to the type of micro-organism they are effective against. For example, antibacterial substances are effective against bacteria, antifungal substances are effective, against fungi, including yeast, and antiviral substances are effective against viruses. Certain antimicrobials can be used internally, for example antibiotic medications, whereas other antimicrobials are for external use only, such as antiseptics.

In a preferred aspect, the cyclic compound of the invention is a compound having formula I, or a derivative thereof,

wherein R¹ and R² are independently selected from —OH, ═O;

wherein R³ is a substituent comprising an —OH group;

wherein R⁴ and R⁵ are each independently selected from a hydrocarbyl group, H, OH or ═O, or represent a bond with an adjacent atom on the ring of the cyclic compound.

In a more preferred aspect, the cyclic compound of the invention is a compound having Formula II

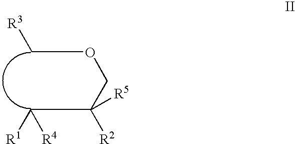

or a derivative thereof, wherein R¹, R², R³, R⁴, and R⁵ are as defined above.

In a further preferred aspect, the cyclic compound of the invention is a compound having Formula III

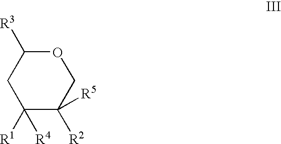

or a derivative thereof; wherein R¹, R², R³, R⁴, and R⁵ are as defined above.

In one preferred aspect of the invention the groups R⁴ and R⁵ of the general formula may independently be a hydrocarbyl group.

The term “hydrocarbyl group” as used herein means a group comprising at least C and H and may optionally comprise one or more other suitable substituents. Examples of such substituents may include halo-, alkoxy-, nitro-, hydroxy, carboxyl, epoxy, acrylic, hydrocarbon, N-acyl, or cyclic group etc. In addition to the possibility of the substituents being a cyclic group, a combination of substituents may form a cyclic group. If the hydrocarbyl group comprises more than one C then those carbons need not necessarily be linked to each other. For example, at least two of the carbons may be linked via a suitable element or group. Thus, the hydrocarbyl group may contain hetero atoms. Suitable hetero atoms will be apparent to those skilled in the art and include, for instance, sulphur, nitrogen and oxygen.

The groups R⁴ and R⁵ of the general formula may independently be selected from alkyl, alkenyl, cycloalkyl and aryl or may together represent an alkylene.

In a particularly preferred aspect of the invention, the derivative of the compound of Formula I is an ester. The term “ester” includes mono-, di-, tri- and poly-esters.

Preferably, the derivative of the compound of formula I is an ester wherein an ester linkage is formed from the —OH group of the R³ substituent. In this aspect preferably the derivatised R³ substituent is a group of the formula —(CH₂)ₙ—OC(O)—(CH₂)ₚCH₃, wherein n and p are independently of each other from 1 to 24, preferably from 1 to 20, preferably from 1 to 10, preferably from 1 to 5, or preferably 1, 2, or 3. In yet a further preferred embodiment the derivatised R³ substituent is a group of the formula —CH₂—OC(O)—(CH₂)ₚCH₃, wherein p is from 1 to 24, preferably from 1 to 20, or p is from 1 to 10, or p is from 1 to 5, and n=1, 2, or 3.

Preferably, the derivative of the compound of formula I is an ester wherein the R¹ substituent and/or the R² substituent is an —OH group and wherein an ester linkage is formed from the —OH group of the R¹ substituent and/or the R² substituent. In this aspect preferably the derivatised R¹ substituent and/or the R² substituent is a group of the formula —(CH₂)ₙ—OC(O)—(CH₂)ₚCH₃, wherein n and p are independently of each other from 1 to 24, preferably from 1 to 20, preferably from 1 to 10, preferably from 1 to 5, or preferably 1, 2, or 3. In yet a further preferred embodiment the derivatised R¹ substituent and/or the R² substituent is a group of the formula —CH₂—OC(O)—(CH₂)ₚCH₃, wherein p is from 1 to 24, preferably from 1 to 20, or p is from 1 to 10, or p is from 1 to 5, and n=1, 2, or 3.

Preferably, the derivative of the compound of formula I is an ester wherein the R¹ substituent and/or the R² substituent is an —OH group and wherein an ester linkage is formed from the —OH group of the R¹ substituent and/or the R² substituent. In this aspect preferably the derivatised R¹ substituent and/or the R² substituent is a group of the formula —OC(O)—(CH₂)ₚCH₃, wherein p is from 1 to 24, preferably from 1 to 20, preferably from 1 to 10, preferably from 1 to 5, or preferably 1, 2, or 3.

In a preferred aspect the compound of formula I is a diester wherein the R¹ substituent is an —OH group and wherein the ester linkages are formed from the —OH group of the R⁴ substituent and from the —OH group of the R³ substituent.

In a highly preferred aspect a derivative of the compound of formula I is a compound of the formula

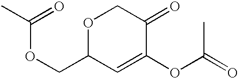

This compound (3,6-di-O-acetyl-1,5-anhydro-4-deoxy-D-glycero-hex-3-enopyranose-2-ulose) may be prepared in accordance with the teaching of Andersen et al. (1998), Structure of 1,5-anhydro-D-fructose: X-ray analysis of crystalline acetylated dimeric forms, J. Carbohydr. Chem. 17: 1027-1035.

The aspect of the present invention wherein the derivative of the compound of formula I is an ester is particularly preferred because the compound may be lipophilic and/or may have both hydrophobic and hydrophilic properties. When the compound has both hydrophobic and hydrophilic properties the compound readily resides at a water/oil interface of an emulsion.

The residence of the compound at a water/oil interface of an emulsion may allow it to act as an emulsifier. Thus the present invention may further provide compounds having a dual functional effect. The compounds may act both as an antimicrobial and as an emulsifier.

In particularly preferred aspect of the invention, the cyclic compound is selected from Ascopyrone P, Ascopyrone M, Ascopyrone T, Ascopyrone T₁, Ascopyrone T₂, and Ascopyrone T₃, and mixtures thereof, the structures of which are shown below.

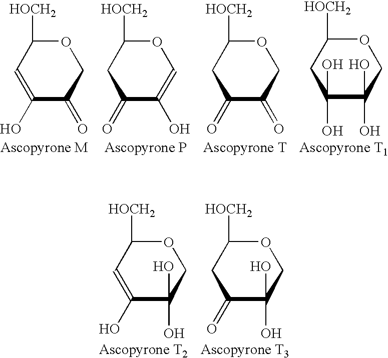

In an especially preferred embodiment, the cyclic compound is ascopyrone P.

Ascopyrone is a known compound. In 1978 and 1981, a group of American scientists prepared Ascopyrone P by pyrolysis of amylopectin, amylose and cellulose at the Wood Chemistry laboratory in Montana, with the intention of using Ascopyrone P as a starting material for organic synthesis [Shafizadeh, F., Furneaux R. H., Stevenson, T. T., and Cochran, T. G., 1,5-Anhydro-4-deoxy-D-glycero-hex-1-en-3-ulose and other pyrolysis products of cellulose, Carbohydr. Res. 67(1978): 433-447; Stevenson, T. T., Stenkmap, R. E., Jensen, L. H., Cochran, T. T., Shafizadeh, F., and Furneaux R. H., The crystal structure of 1,5-anhydro-4-deoxy-D-glycero-hex-1-en-3-ulose, Carbohydr. Res. 90(1981): 319-325]. They characterised Ascopyrone P by, for example, ¹H and ¹³C NMR, and IR spectroscopy techniques. A 3-dimensional structure of Ascopyrone P was provided. The yield of Ascopyrone P obtained by pyrolysis was under 3% and complicated separation methods had to be used.

The natural occurrence of Ascopyrone P in some species of very scarcely studied fungi collected from the Alps has been taught [M.-A. Baute, G. Deffieux, J. Vercauteren, R. Baute, and Badoc A., Enzymatic activity degrading 1,4-α-glucans to Ascopyrones P and T in Pezizales ad Tuberales, Phytochemistry, 33 (1993): 41-45]. The occurrence of Ascopyrone P in fungi immediately prompted the hypothesis that Ascopyrone P would act as an antibiotic. However, Ascopyrone P did not function satisfactorily as an antibiotic in the disclosed tests.

Many of the compounds of the present invention can be derived from 1,5-anhydrofructose. 1,5-Anhydrofructose is monoketo sugar found in bacteria, red algae, fungi and mammals. In red algae and fungi 1,5-anhydrofructose is produced by the action of α-1,4-glucan lyase [EC 4.2.2.13] from floridean starch and glycogen, respectively.

When the compound of the present invention is prepared from 1,5-anhydro-D-fructose, preferably the 1,5-anhydro-D-fructose is prepared in accordance with GB-A-2296717. In other words, preferably the 1,5-anhydro-D-fructose is prepared by a method comprising treating an α-1,4-glucan with the enzyme α-1,4-glucan lyase characterised in that enzyme is used in substantially pure form.

Ascopyrone P and Ascopyrone T can be produced enzymatically from 1,5-anhydro-D-fructose using cell-free extract prepared from the fungi of the order Pezizales, such as Plicaria leiocarpa and Anthracobia melaloma, and the order of Tuberales, such as, Tuber melanosporum. Ascopyrone T₁ is the dihydrate form of Ascopyrone T, whereas Ascopyrone T₂ and T₃ are the tautomeric monohydrate forms of Ascopyrone T.

Ascopyrone M can be produced from 1,5-anhydro-D-fructose by EDTA-sensitive dehydratases isolated from the fungi Morels, such as Morchella vulgaris, Gyromitres, pezizes, such as Peziza echinospora.

Ascopyrone M, P and T can also be produced chemically by treating 1,5-anhydro-D-fructose with alkali under mild conditions [Studies on the degradation of some pentoses and of 1,5-anhydro-D-fructose, the product of the starch-degrading enzyme a-1,4-glycan lyase; Thesis, Ahmad, T., The Swedish University of Agricultural Sciences, Sweden, 1995].

When the compound of the present invention is prepared by chemical means, it may be prepared in accordance with one of the following methods:

(1) Ascopyrone P may be produced by treating 1,5-anhydro-D-fructose with non-aqueous acid at elevated temperature, for example at 70° C.

(2) Ascopyrones (for example, Ascopyrone P, T and M) may be produced from 1,5-anhydro-D-fructose by alkaline treatment according to Ahmad, T., 1995.

The structures of all ascopyrones produced were confirmed by NMR techniques.

Preferably, the compound of the present invention is prepared by enzymatic means as disclosed in M.-A. Baute et al, [Phytochemistry, 33 (1993): 41-45). For example ascopyrones (such as, Ascopyrone P, T and M) may be produced from 1,5-anhydro-D-fructose using enzymatic methods as disclosed in M.-A. Baute et al.

In another preferred aspect, the cyclic compound of the invention is of Formula IV,

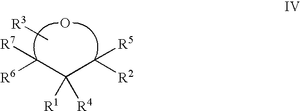

or a derivative thereof,

wherein R¹ and R² are independently selected from —OH, ═O, and OR′, wherein R′ is H or —COR″, and R″ is C₁₋₁₀alkyl;

wherein R³ is a substituent comprising an —OH group;

wherein R⁴ and R⁵ are each independently selected from a hydrocarbyl group, H, OH or ═O, or represent a bond with an adjacent atom on the ring of the cyclic compound;

wherein R⁶ and R⁷ are each independently selected from H, OH or ═O, or represent a bond with an adjacent atom on the ring of the cyclic compound.

In a more preferred aspect, the cyclic compound of the invention is of formula V,

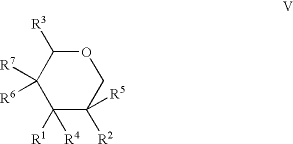

or a derivative thereof, wherein R¹, R², R³, R⁴, R⁵, R⁶ and R⁷ are as defined above.

Even more preferably, the cyclic compound of the invention is selected from one or more of the following:

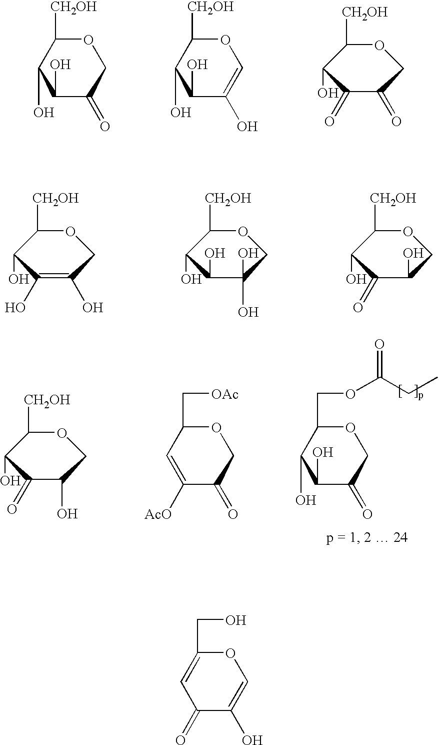

In a particularly preferred aspect of the invention, R³ is or comprises a CH₂OH group.

Preferably, the cyclic compound of the invention comprises a five or a six membered ring.

In respect of said third, fourth and fifth aspects of the invention, the cyclic compound may be used alone, or in combination with other components, for example, one or more chelators (such as EDTA sodium salt, polyphosphate or citrate) and/or one or more antioxidants (such as ascorbate, isoascorbate, ascorbate palmitate, BHA or BHT).

Tests indicate that APP is stable in water at 24° C. for 2 weeks, but completely disappears after 2 months. It has been reported that APP is stable at pH 1.5-5.5, and less stable at increasing pH. At pH 11-12.5 APP has a half-life of 5 h. Since many materials are acidic or neutral, the stability may therefore be improved by using APP in combination with an antioxidant such as those listed hereinbefore.

In respect of said third, fourth and fifth aspects of the invention, in one preferred embodiment, the compound is used in combination with one or more preservatives. By way of definition, in the broadest sense, the term “preservative” is intended to encompass all substances which inhibit the development of, or kill, micro-organisms. In a narrower sense, it is generally understood that preservatives are used in concentrations of 0.5% or less. Food additives which are allowed to be used as preservatives are listed in the Regulation No. 95/2/EG of the European Parliament and Council of 20 February 1995, relating to food additives other than colouring agents and sweeteners.

Typical food preservatives permitted in the EU which are suitable for use in combination with the compounds of the invention include sorbic acid, benzoic acid, PHB ester (p-hydroxybenzoate), and sulphur dioxide. The mode of action of these preservatives, together with their range of effects are listed below.

Sorbic Acid (E200 to 203):

Mode of action: inhibits different enzymes in the cells of the micro-organisms.

Range of effects: mainly against yeasts and moulds as well as catalase-positive bacteria. Catalase-negative bacteria as well as lactic acid bacteria and clostridia are not inhibited.

Effective concentration: 500-3000 ppm.

Permitted maximum quantities in food: up to 2000 ppm in potato dough, processed cheese, packed bread, fine bakery products, emulsified sauces etc.

Benzoic Acid (E210 to 213):

Mode of action: inhibits exchange of oxygen through the cellular membrane and affects the enzymatic structure.

Range of effects: for acid products only, up to approx. pH 4.5; inhibits yeasts and moulds, restricted inhibition of bacteria (no, or only very little, inhibition of lactic acid bacteria and clostridia).

Permitted maximum quantities in food: 500 ppm in aspic, fruit preparations, marmalades etc.

PHB Ester (p-hydroxybenzoate) (E214 to 219)

Mode of action: damages the bacterial membrane because of the surface activity, poisonous to protoplasm because of protein denaturation.

Range of effects: mainly inhibits yeasts and fungi, but also Gram-positive bacteria in a pH range between 3.0 and 8.0.

Effective concentration: sensorical influence at concentrations beyond approx. 0.08%.

Sulphur Dioxide (E220 to 224; E 226 to 227)

Mode of action: depends on pH to a great extent, in practice it is only effective at acidic pH values (<4,0). Very complex mechanisms.

Range of effects: mainly antibacterial, above all against Gram-negative, aerobic bacteria.

Effective concentrations: 250-500 ppm for inhibition of aerobic, Gram-negative bacteria, 800-2000 ppm against Gram-positive bacteria, yeasts, and moulds.

Permitted maximum quantity in food products: max. 2000 ppm in dry fruits, grape juice concentrate for home production of wine, in some cases only max. quantities of 20-30 ppm are permitted.

For more specific applications, the compounds of the present invention may also be used in combination with the following preservatives: biphenyl, diphenyl, orthophenylphenol, thiabendazol, nisin, natamycin, hexamethylentetramine, dimethyldicarbonate, boric acid, sodiumtetraborate, nitrite, propionic acid and propionate, and lysozyme. The mode of action of these preservatives, together with their range of effects and specific uses are listed below.

Biphenyl, Diphenyl (E 230)

Range of effects: Inhibition of moulds.

Substance for treatment of fruits: surface treatment of citrus fruits.

Permitted maximum quantity: 70 ppm

Orthophenylphenol (E 231/E 232)

As with E230, limited to treatment of fruits as a surface treatment for citrus fruits.

Thiabendazol (E 233)

Surface treatment of citrus fruits and bananas.

Nisin (E 234)

Mode of action: Disturbance of membrane functions.

Range of effects: Gram-positive bacteria, no influence on Gram-negative bacteria.

Permitted maximum quantity in food products (EU): 3 ppm in semolina pudding and similar products, 12.5 ppm (=12.5 IU/g) in ripened cheese and processed cheese, 10 ppm in clotted cream, 10 ppm in mascarpone.

Natamycin (Pimaricin) (E235)

Mode of action: specifically attacks cell membrane, where—in general—an interaction with sterines occurs which increases the permeability of the membrane.

Range of effects: Moulds and yeasts, not effective against bacteria. Usual dosage rates are below approx. 50 mg/1. Maximum level is 1 mg/dm² on the surface, with a maximum penetration of 5 mm.

Applications: surface treatment of hard, semi-hard and semi-soft cheese and of dried, cured sausages.

Hexamethylentetramine (E 239)

Hexamethylentetramine is formed by adding ammonia to formaldehyde in an aqueous solution. The microbicidal effect is due to the formaldehyde.

Permitted only for Provolone cheese (25 ppm residual quantity).

Dimethyldicarbonate (E 242)

Permitted only for non-alcoholic drinks, non-alcoholic wine, and liquid concentrate.

Boric Acid, Sodiumtetraborate (E284/E 285)

Permitted only for caviar.

Nitrite (E 249 and E 250)

Permitted in the form of nitrite curing salt for treatment of meat products (“red products”). For cured and dried meat products which are not heat treated and for other cured meat products an addition of 150 ppm has been fixed as a guideline. These concentrations do not show a preservative effect. They are mainly added for their technological properties (formation of colour, taste) as well as for their antioxidant effects.

Propionic Acid and Propionate (E 280, E 281, E 282, and E 283)

Mode of action: similar to sorbic acid, pH<4.5 is optimal.

Accumulation in the cell leads to inhibition of enzymes.

Range of inhibition: moulds are inhibited at an pH of 5.5 by concentrations of 125 to 12500 ppm, for inhibition of bacteria higher concentrations are necessary (>16000 ppm).

Application: Sliced and packaged bread.

Permitted maximum quantity: 3000 ppm.

Lysozyme (E 1105)

Permitted only for ripened cheese.

Permitted maximum quantity: quantum satis.

Studies by the applicant of the inhibitive effects of the present compounds have been tested in a medium (Elliker broth) with an almost neutral pH (pH 6.8) and have been shown to be effective against both Gram-positive and Gram-negative bacteria. As many of the preservatives described above show an inhibitory effect mainly at low pH, the use of the compounds of the present invention clearly broadens the potential range of applications.

In principle, the use of substances for chemical preservation depends on the following factors:

(a) Toxicological Harmlessness

the effects of the substance when applied acutely, subchronically, and for a long term period.

Testing of acute toxicity (LD₅₀), cinetics and metabolism, pharmacological effects, genotoxicity, etc.

(b) Technological/Food Chemical Aspects:

Solubility in water: as growth takes place in the aqueous phase, a preservative has to be water-soluble

Reaction with food ingredients, problem of off-flavours (sensory acceptance)

Interferences with food ingredients (e.g. destruction of vitamin B1 by sulphuric acid)

The antimicrobial effectiveness of chemical substances in food and feed products is thus determined by a range of different factors. Among others, the composition of the population of micro-organisms, the composition of the food product (ingredients, pH, water activity, content of salt, etc.), the packaging, time-temperature-conditions, etc. are key factors that influence the inhibitory activities of the antimicrobial agent.

Pharmaceutical Compositions

A further aspect of the invention provides a pharmaceutical composition comprising a compound of Formula I admixed with a pharmaceutically acceptable diluent, excipient or carrier, or a mixture thereof.

The pharmaceutical compositions may be for human or animal usage in human and veterinary medicine and will typically comprise any one or more of a pharmaceutically acceptable diluent, carrier, or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s).

Examples of suitable carriers include lactose, starch, glucose, methyl cellulose, magnesium stearate, mannitol, sorbitol and the like. Examples of suitable diluents include ethanol, glycerol and water.

Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol.

Examples of suitable lubricants include sodium oleate, sodium stearate, magnesium stearate, sodium benzoate, sodium acetate, sodium chloride and the like.

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

There may be different composition/formulation requirements dependent on the different delivery systems. By way of example, the pharmaceutical composition of the present invention may be formulated to be administered using a mini-pump or by a mucosal route, for example, as a nasal spray or aerosol for inhalation or ingestable solution, or parenterally in which the composition is formulated by an injectable form, for delivery, by, for example, an intravenous, intramuscular or subcutaneous route. Alternatively, the formulation may be designed to be administered by a number of routes.

Administration

The compounds of the present invention may be administered alone but will generally be administered as a pharmaceutical composition—e.g. when the components are is in admixture with a suitable pharmaceutical excipient, diluent or carrier selected with regard to the intended route of administration and standard pharmaceutical practice. For example, the composition can be administered (e.g. orally or topically) in the form of tablets, capsules, ovules, elixirs, solutions or suspensions, which may contain flavouring or colouring agents, for immediate-, delayed-, modified-, sustained-, pulsed- or controlled-release applications.

If the pharmaceutical composition is a tablet, then the tablet may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, cellulose, milk sugar or high molecular weight polyethylene glycols. For aqueous suspensions and/or elixirs, the compound may be combined with various sweetening or flavouring agents, colouring matter or dyes, with emulsifying and/or suspending agents and with diluents such as water, ethanol, propylene glycol and glycerin, and combinations thereof.

The routes for administration (delivery) include, but are not limited to, one or more of: oral (e.g. as a tablet, capsule, or as an ingestable solution), topical, mucosal (e.g. as a nasal spray or aerosol for inhalation), nasal, parenteral (e.g. by an injectable form), gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic (including intravitreal or intracameral), transdermal, rectal, buccal, vaginal, epidural, sublingual.

In one preferred embodiment, the composition comprises more than one compound of Formula I. In this case, it is to be understood that not all of the components of the pharmaceutical need be administered by the same route. Likewise, if the composition comprises more than one active component, then those components may be administered by different routes.

If a compound of the present invention is administered parenterally, then examples of such administration include one or more of: intravenously, intra-arterially, intraperitoneally, intrathecally, intraventricularly, intraurethrally, intrasternally, intracranially, intramuscularly or subcutaneously administering the component; and/or by using infusion techniques.

For parenteral administration, the compound is best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

As indicated, the compound(s) of the present invention can be administered intranasally or by inhalation and is conveniently delivered in the form of a dry powder inhaler or an aerosol spray presentation from a pressurised container, pump, spray or nebuliser with the use of a suitable propellant, e.g. dichlorodifluoromethane, trichlorofluoromethane, dichlorotetrafluoroethane, a hydrofluoroalkane such as 1,1,1,2-tetrafluoroethane (HFA 134A™) or 1,1,1,2,3,3,3-heptafluoropropane (HFA 227EA™), carbon dioxide or other suitable gas. In the case of a pressurised aerosol, the dosage unit may be determined by providing a valve to deliver a metered amount. The pressurised container, pump, spray or nebuliser may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of the agent and a suitable powder base such as lactose or starch.

Alternatively, the compound(s) of the present invention can be administered in the form of a suppository or pessary, or it may be applied topically in the form of a gel, hydrogel, lotion, solution, cream, ointment or dusting powder. The compound(s) of the present invention may also be dermally or transdermally administered, for example, by the use of a skin patch. They may also be administered by the pulmonary or rectal routes. They may also be administered by the ocular route. For ophthalmic use, the compounds can be formulated as micronised suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

For application topically to the skin, the compound(s) of the present invention can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, it can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

In a preferred embodiment of the invention, the pharmaceutical composition is administered orally.

Dose Levels

Typically, a physician will determine the actual dosage which will be most suitable for an individual subject. The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy.

Depending upon the need, the agent may be administered at a dose of from 0.01 to 30 mg/kg body weight, such as from 0.1 to 10 mg/kg, more preferably from 0.1 to 1 mg/kg body weight.

Where the composition is to be administered mucosally through the gastrointestinal mucosa, it should be able to remain stable during transit though the gastrointestinal tract; for example, it should be resistant to proteolytic degradation, stable at acid pH and resistant to the detergent effects of bile.

Where appropriate, the pharmaceutical compositions can be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

The invention will now be described only by way of example, and with reference to the accompanying figures, wherein:

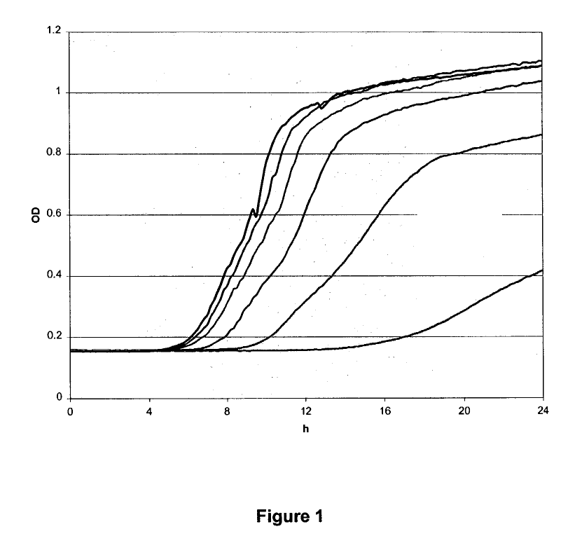

FIG. 1 shows the effect of APP on the growth of B. anthracis at 30° C. over a period of 24 h (optical density versus time in hours).

### EXAMPLES

Trials were undertaken to investigate the antimicrobial efficacy of ascopyrone P (APP) in laboratory media and in a range of systems.

1. Material and Methods

1.1 Test Strains

Micro-organisms are taken from storage at −80° C. Bacillus species are tested as endospore suspensions prepared earlier and stored at 4° C. For Bioscreen testing the bacteria are grown in Brain Heart Infusion (BHI, Oxoid, pH 7.4) and cultured at 30° C.

1.2 Ascopyrone P (APP) Samples

Sample E002012 is a dry powder that was dissolved in sterile de-ionised water. It is tested without filtration and used in the mini well diffusion test, initial Bioscreen runs (BS1211200; BS131200), and mini cidal experiment. The concentration of this sample is 49.3 mg/ml. Further APP samples are prepared as follows: 3.84 g of batch number APP20010213, as 5×4 ml volumes, is made up to a concentration of 169 mg APP/ml, and 5.5 g of batch number APP20010215, as 4×10 ml volumes, is made up to a concentration of 138 mg APP/ml. All samples are kept at −20° C. until use. Stock solutions are made up in de-ionised water and filter sterilised. These samples are used in Bioscreen run BS010411 and BS010420. APP20010215 is used in Bioscreen run BS010510.

1.3 In Vitro Growth Inhibition Test Methods Testing

1.3.1 Well Diffusion Testing

Seeded 10 ml agar plates of the test organism are prepared, with inoculation of 20 μl spore suspension. This gives an inoculum level of ca. 10⁵-10⁶ cfu/ml. After the plates have set, small wells are cut, and 20 μl of APP sample is loaded. Plates are incubated overnight at appropriate temperature and examined after 1-2 days (after which microbial growth is clearly visible) for zones of inhibition.

1.3.2 Bioscreen Testing

An automated Microbiology Reader Bioscreen C is used to measure growth curves of the strains in the presence and absence of APP. The Bioscreen C measures the development of turbidity (i.e. growth) kinetically by vertical photometry in 200 wells of a honeycomb microtitre plate, simultaneously. The system consists of a Bioscreen C analyser, which is an incubator and measurement unit, integrated with a PC, software (BioLink v 5.30), printer and a ‘Honeycomb 2’ cuvette multiwell plate. Growth curve data can be analysed within the BioLink software or exported to programs such as Excel.

Broth culture media are dispensed in 270 μl volumes into the wells as directed. Serial dilutions of a filter-sterilised APP stock solution are then dispensed into the same wells, as appropriate. The wells are inoculated with 30 μl of an appropriately diluted spore suspension, to give a final inoculum level of ca. 10³ cfu/ml. The tests are incubated in the Bioscreen C for either 24 h at 30° C., or 72 h at 25° C. with readings taken every 20 minutes after the trays are shaken. After the incubation period is complete the data are exported to Excel for analysis.

2. Results

2.1 In Vitro Growth Inhibition Results

2.1.1. Well Diffusion Results

The concentration of the test solution is 49.3 mg/ml (4.9%). Bacillus anthracis is sensitive to this level.

2.1.2. Bioscreen Test Results: in vitro Sensitivity of Bacillus Anthracis

The Bioscreen test results are summarised in Tables 1 below and FIG. 1.

TABLE 1
Bioscreen results: effective levels of APP against Gram-positive bacteria

| | APP LEVEL (ppm) CAUSING SIGNIFICANT OR TOTAL | | | | | |
|---|---|---|---|---|---|---|
| | INHIBITION FOR TIME/TEMPERATURE INDICATED | | | | | |
| GRAM | BS010510 | | BS010411 | | BS010411 | |
| POSITIVE | 24 h at 30° C. | | 24 h at 30° C. | | 24 h at 30° C. | |
| [Bacillus tested | [APP20010213] | | [APP20010213] | | [APP20010215] | |
| as spores] | Total | Significant | Total | Significant | Total | Significant |
| B. anthracis 204 | | | >4000 | 4000 | 4000 | 2000 |
| B. anthracis | | | 4000 | 2000 | >4000 | 2000 |
| B. anthracis 3.046 | 3000 | 2000 | | | | |

3. Applications

3.1 Skin Cream (Oil-in-Water)

| Ingredients | Amount (%) |
|---|---|
| Butane-1,3-diol | 13.5 |
| Xanthan Gum (Rhodopol 23) | 0.5 |
| Ozokerite Wax | 2.56 |
| Glyceryl Monostearate | 3.84 |
| Hexadecanol | 0.6 |
| Titanium Diowide | 0.15 |
| Volatile Silicone (DC345) | 7.6 |
| Ethoxylated Emulsifier (Brij 58) | 1.4 |
| Sodium Hydroxide (to pH 5.5) | q.s. |
| Demineralised Water | to 100 |

To examine the preserving effect of APP, AF-ester and AF in skin cream, cream were prepared by the recipe above. Cream samples were added either a normally used preservative (methyl paraben) in sufficient amount or APP or AF-ester or AF in the concentrations: 250 or 2000 ppm.

The results showed that APP, AF-ester and AF in both concentrations were as good preservatives as the commonly used preservative. Addition of APP, AF-ester and AF to the cream showed no irritation effect on the skin.

3.2 Shampoo

| Ingredients | Amount (%) |
|---|---|
| 2-hydroxy octanoic acid | 1 |
| Sodium lauryl ether sulphate | 11 |
| Lauryl dimethyl betaine | 2 |
| Coconut diethanolamide | I |
| Electrolyte (to adjust viscosity) | 1.3 |
| Citric Acid (to pH 6.5) | q.s. |
| Demineralised water | to 100 |

To examine the preserving effect of APP, AF-ester and AF in shampoo, a shampoo according to the above-mentioned recipe were prepared. Samples of shampoo were added respectively a normally used preservative in sufficient amount and APP or AF-ester or AF in the concentration 2000 ppm.

The results showed that APP, AF-ester and AF were as good preservatives as the commonly used preservative. Addition of APP, AF-ester and AF to the shampoo showed no irritation effect on the scalp.

3.3 Toothpaste

| Ingredients | Amount (%) |
|---|---|
| Glycerine | 22.0 |
| Dicalcium phosphate | 49.0 |
| Sodium lauryl sulfate | 2.0 |
| Sodium saccharin | 0.2 |
| Sodium monofluorophosphate | 0.75 |
| Tetrasodium pyrophosphate | 0.25 |
| Thickening agent | 1.0 |
| Colour and flavour | to suit |
| Demineralised water | to 100.0 |

To examine the preserving effect of APP, AF-ester and AF in toothpaste, toothpaste according to the above-mentioned recipe were prepared. Toothpaste samples were added respectively a normally used preservative (sodium benzoate) in sufficient amount and APP or AF-ester or AF in the concentration 2000 ppm.

The results showed that APP, AF-ester and AF were as good preservatives as the commonly used preservative. Addition of APP to the toothpaste shoved that the APP inhibited caries.

Various modifications and variations of the described methods and system of the invention will be apparent to those skilled in the art without departing from the scope and spirit of the invention. Modifications of the described modes for carrying out the invention which are obvious to those skilled in the relevant are, or related fields, are thus intended to fall within the scope of the following claims.

The invention will now be further described by the following numbered paragraphs:

1. Use of a cyclic compound having Formula I in medicine,

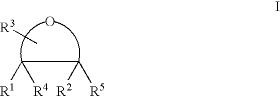

or a derivative thereof,

wherein R¹ and R² are independently selected from —OH, ═O, and OR′, wherein R′ is H or —COR″, and R″ is C₁₋₁₀alkyl;

wherein R³ is a substituent comprising an —OH group;

wherein R⁴ and R⁵ are each independently selected from a hydrocarbyl group, H, OH or ═O, or represent a bond with an adjacent atom on the ring of the cyclic compound.

2. Use of a compound having Formula I, or a derivative thereof,

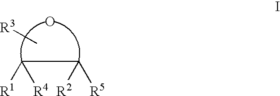

or a derivative thereof,

wherein R¹ and R² are independently selected from —OH, ═O, and OR′, wherein R′ is H or —COR″, and R″ is C₁₋₁₀alkyl;

wherein R³ is a substituent comprising an —OH group;

wherein R⁴ and R⁵ are each independently selected from a hydrocarbyl group, H, OH or ═O, or represent a bond with an adjacent atom on the ring of the cyclic compound;

in the preparation of a medicament for the treatment of a condition associated with the presence of one or more microrganisms.

3. Use according to paragraph 2 wherein said microorganism is selected from Listeria, Salmonella, Bacillus, Saccharomyces, Pseudomonas, Clostridium, Lactobacillus, Brochothrix, Micrococcus, Yersinia, Enterobacter, Escherichia, Zygosaccharomyces and Staphylococcus.

4. Use according to paragraphs 2 or paragraph 3 wherein said microorganism is selected from Listeria monocytogenes, Listeria innocua, Salmonella Typhimurium, Salmonella sp., Bacillus cereus, Bacillus subtilis, Saccharomyces cerevisiae, Saccharomyces cerevisiae var. paradoxus, Saccharomyces carlsbergensis, Pseudomonas fluorescens, Clostridium sporogenes, Lactobacillus sake, Brochothrix thermosphacta, Micrococcus luteus, Yersinia enterocolitica, Enterobacter aerogenes, E. coli, Staphylococcuc aureus, Bacillus anthracis and Zygosaccharomyces bailii.

5. The invention according to any one of paragraphs 2 to 4 wherein said microorganism is Bacillus anthracis.

6. An antimicrobial composition for use against Bacillus anthracis, said composition comprising a cyclic compound having Formula I,

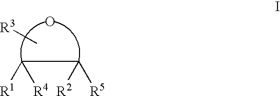

or a derivative thereof,

wherein R¹ and R² are independently selected from —OH, ═O, and OR′, wherein R′ is H or —COR″, and R″ is C₁₋₁₀alkyl;

wherein R³ is a substituent comprising an —OH group;

wherein R⁴ and R⁵ are each independently selected from a hydrocarbyl group, H, OH or ═O, or represent a bond with an adjacent atom on the ring of the cyclic compound.

7. A process for preventing and/or inhibiting the growth of, and/or killing Bacillus anthracis in a material, the process comprising the step of contacting the material with a cyclic compound having Formula I,

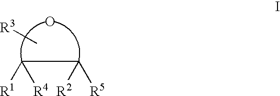

or a derivative thereof,

wherein R¹ and R² are independently selected from —OH, ═O, and OR′, wherein R′ is H or —COR″, and R″ is C₁₋₁₀alkyl;

wherein R³ is a substituent comprising an —OH group;

wherein R⁴ and R⁵ are each independently selected from a hydrocarbyl group, H, OH or ═O, or represent a bond with an adjacent atom on the ring of the cyclic compound.

8. Use of a compound having Formula I, or a derivative thereof,

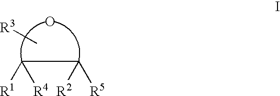

or a derivative thereof,

wherein R¹ and R² are independently selected from —OH, ═O, and OR′, wherein R′ is H or —COR″, and R″ is C₁₋₁₀alkyl;

wherein R³ is a substituent comprising an —OH group;

wherein R⁴ and R⁵ are each independently selected from a hydrocarbyl group, H, OH or ═O, or represent a bond with an adjacent atom on the ring of the cyclic compound;

for preventing and/or inhibiting the growth of, and/or killing Bacillus anthracis in a material.

9. The invention according to paragraph 7 or paragraph 8 wherein said material is a foodstuff.

10. The invention according to paragraph 7 or paragraph 8 wherein said material is a non-food material.

11. Use according to paragraph 8 in surface cleaning, laundry or in a cosmetic or pharmaceutical product.

12. The invention of any one of the preceding paragraphs wherein said cyclic compound is a compound having formula I, or a derivative thereof,

wherein R¹ and R² are independently selected from —OH, ═O;

wherein R³ is a substituent comprising an —OH group;

wherein R⁴ and R⁵ are each independently selected from a hydrocarbyl group, H, OH or ═O, or represent a bond with an adjacent atom on the ring of the cyclic compound.

13. The invention of any one of the preceding paragraphs wherein the cyclic compound is a compound having Formula II

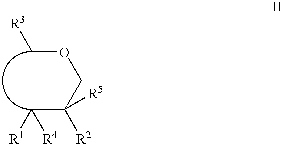

or a derivative thereof, wherein R¹, R², R³, R⁴, and R⁵ are as defined in the preceding paragraphs.

14. The invention of any one of the preceding paragraphs wherein the cyclic compound is a compound having Formula III

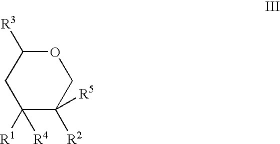

or a derivative thereof, wherein R¹, R², R³, R⁴, and R⁵ are as defined in the preceding paragraphs.

15. The invention of any one of the preceding paragraphs wherein the compound is selected from Ascopyrone P, Ascopyrone M, Ascopyrone T, Ascopyrone T₁, Ascopyrone T₂, Ascopyrone T₃, and mixtures thereof.

16. The invention according to any preceding paragraph wherein said compound is ascopyrone P.

17. The invention of any one of paragraphs 1 to 12 wherein said cyclic compound is of Formula IV,

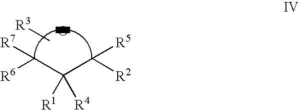

or a derivative thereof,

wherein R¹ and R² are independently selected from R³, —OH, ═O, and OR′, wherein R′ is H or —COR″, and R″ is C₁₋₁₀alkyl;

wherein R³ is a substituent comprising an —OH group;

wherein R⁴ and R⁵ are each independently selected from a hydrocarbyl group, H, OH or ═O, or represent a bond with an adjacent atom on the ring of the cyclic compound;

wherein R⁶ and R⁷ are each independently selected from H, OH or ═O, or represent a bond with an adjacent atom on the ring of the cyclic compound.

18. The invention of paragraph 17 wherein said cyclic compound is of formula V,

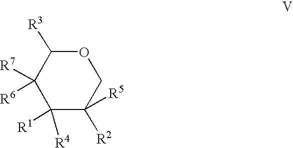

or a derivative thereof, wherein R¹, R², R³, R⁴, R⁵, R⁶ and R⁷ are as defined in paragraph 15.

19. The invention according to any one of the preceding paragraphs wherein the derivative of the compound of formula I is an ester.

20. The invention according to paragraph 19 wherein the ester is formed from an —OH substituent on the cyclic compound, and wherein said ester is of the formula —(CH₂)ₙ—OC(O)—(CH₂)ₚCH₃, wherein n and p are each independently from 1 to 24.

21. The invention of any one of paragraphs 17 to 20 wherein said cyclic compound is selected from one or more of the following:

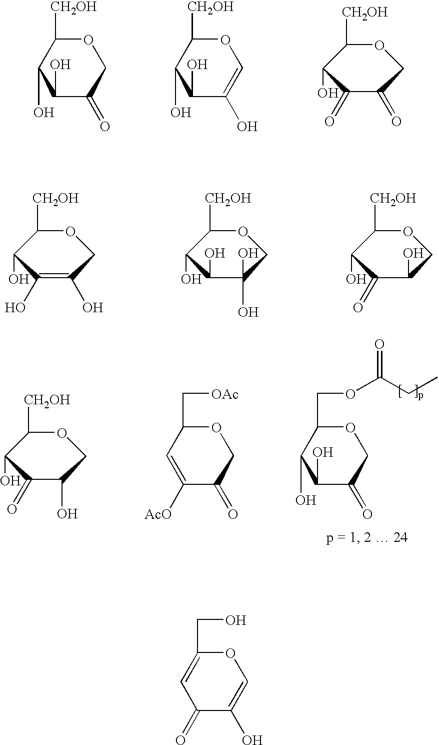

22. The invention of any one of the preceding paragraphs wherein R³ is or comprises a CH₂OH group.

23. The invention of any one of the preceding paragraphs wherein the cyclic compound comprises a five or a six membered ring.

24. The invention according to any preceding paragraph wherein said compound of formula I is used in combination with one or more of an antioxidant, a preservative and/or a chelator.

25. A pharmaceutical composition comprising

(i) a compound a Formula I,

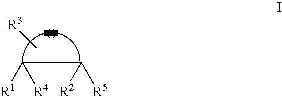

or a derivative thereof,

wherein R¹ and R² are independently selected from —OH, ═O, and OR′, wherein R′ is H or —COR″, and R″ is C₁₋₁₀alkyl;

wherein R³ is a substituent comprising an —OH group;

wherein R⁴ and R⁵ are each independently selected from a hydrocarbyl group, H, OH or ═O, or represent a bond with an adjacent atom on the ring of the cyclic compound; and

(ii) a pharmaceutically acceptable diluent, excipient or carrier, or a mixture thereof.

1. A method of treatment comprising administering a cyclic compound having Formula I, 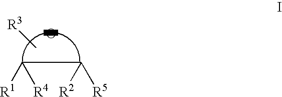
or a derivative thereof,
wherein R¹ and R² are independently selected from —OH, ═O, and OR′, wherein R′ is H or —COR″, and R″ is C₁₋₁₀alkyl;
wherein R³ is a substituent comprising an —OH group;
wherein R⁴ and R⁵ are each independently selected from a hydrocarbyl group, H, OH or ═O, or represent a bond with an adjacent atom on the ring of the cyclic compound.

2. The method of claim 1, for the treatment of a condition associated with the presence of one or more microrganisms.

3. The method of claim 2 wherein said microorganism is selected from Listeria, Salmonella, Bacillus, Saccharomyces, Pseudomonas, Clostridium, Lactobacillus, Brochothrix, Micrococcus, Yersinia, Enterobacter, Escherichia, Zygosaccharomyces and Staphylococcus.

4. The method of claim 3 wherein said microorganism is selected from Listeria monocytogenes, Listeria innocua, Salmonella Typhimurium, Salmonella sp., Bacillus cereus, Bacillus subtilis, Saccharomyces cerevisiae, Saccharomyces cerevisiae var. paradoxus, Saccharomyces carlsbergensis, Pseudomonas fluorescens, Clostridium sporogenes, Lactobacillus sake, Brochothrix thermosphacta, Micrococcus luteus, Yersinia enterocolitica, Enterobacter aerogenes, E. coli, Staphylococcuc aureus, Bacillus anthracis and Zygosaccharomyces bailii.

5. The method of claim 4 wherein said microorganism is Bacillus anthracis.

6. An antimicrobial composition for use against Bacillus anthracis, said composition comprising a cyclic compound having Formula I, 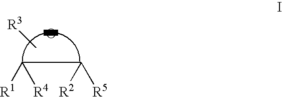
or a derivative thereof,
wherein R¹ and R² are independently selected from —OH, ═O, and OR′, wherein R′ is H or —COR″, and R″ is C₁₋₁₀alkyl;
wherein R³ is a substituent comprising an —OH group;
wherein R⁴ and R⁵ are each independently selected from a hydrocarbyl group, H, OH or ═O, or represent a bond with an adjacent atom on the ring of the cyclic compound.

7. A method of preventing and/or inhibiting the growth of, and/or killing Bacillus anthracis in a material, the process comprising the step of contacting the material with the composition of claim 6.

8. The method of claim 7 wherein said material is a foodstuff.

9. The method of claim 7 wherein said material is a non-food material.

10. The composition of claim 6, for use in surface cleaning, laundry or in a cosmetic or pharmaceutical product.

11. The method of claim 1, wherein said cyclic compound is a compound having formula I, or a derivative thereof,
wherein R¹ and R² are independently selected from —OH, ═O;
wherein R³ is a substituent comprising an —OH group;
wherein R⁴ and R⁵ are each independently selected from a hydrocarbyl group, H, OH or ═O, or represent a bond with an adjacent atom on the ring of the cyclic compound.

12. The composition of claim 6, wherein said cyclic compound is a compound having formula I, or a derivative thereof,
wherein R¹ and R² are independently selected from —OH, ═O;
wherein R³ is a substituent comprising an —OH group;
wherein R⁴ and R⁵ are each independently selected from a hydrocarbyl group, H, OH or ═O, or represent a bond with an adjacent atom on the ring of the cyclic compound.

13. The method of claim 1, wherein the cyclic compound is a compound having Formula II 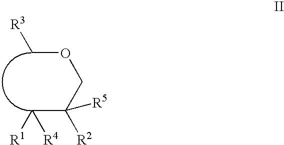
or a derivative thereof, wherein R¹, R², R³, R⁴, and R⁵ are as defined in the preceding claims.

14. The composition of claim 6, wherein the cyclic compound is a compound having Formula II 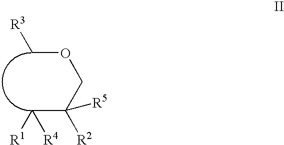
or a derivative thereof, wherein R¹, R², R³, R⁴, and R⁵ are as defined in the preceding claims.

15. The method of claim 1, wherein the cyclic compound is a compound having Formula III 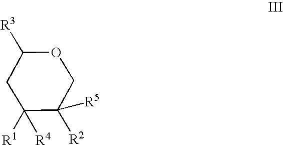
or a derivative thereof, wherein R¹, R², R³, R⁴, and R⁵ are as defined in the preceding claims.

16. The composition of claim 6, wherein the cyclic compound is a compound having Formula III 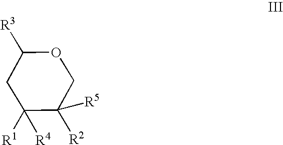
or a derivative thereof, wherein R¹, R², R³, R⁴, and R⁵ are as defined in the preceding claims.

17. The method of claim 1, wherein the compound is selected from Ascopyrone P, Ascopyrone M, Ascopyrone T, Ascopyrone T₁, Ascopyrone T₂, Ascopyrone T₃, and mixtures thereof.

18. The composition of claim 6, wherein the compound is selected from Ascopyrone P, Ascopyrone M, Ascopyrone T, Ascopyrone T₁, Ascopyrone T₂, Ascopyrone T₃, and mixtures thereof.

19. The method of claim 17, wherein said compound is ascopyrone P.

20. The composition of claim 18, wherein said compound is ascopyrone P.

21. The method of claim 1, wherein said cyclic compound is of Formula IV, 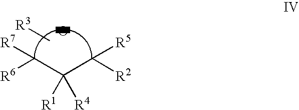
or a derivative thereof,
wherein R¹ and R² are independently selected from R³, —OH, ═O, and OR′, wherein R′ is H or —COR″, and R″ is C₁₋₁₀alkyl;
wherein R³ is a substituent comprising an —OH group;
wherein R⁴ and R⁵ are each independently selected from a hydrocarbyl group, H, OH or ═O, or represent a bond with an adjacent atom on the ring of the cyclic compound;
wherein R⁶ and R⁷ are each independently selected from H, OH or ═O, or represent a bond with an adjacent atom on the ring of the cyclic compound.

22. The composition of claim 6, wherein said cyclic compound is of Formula IV, 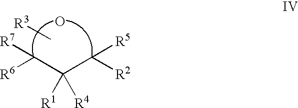
or a derivative thereof,
wherein R¹ and R² are independently selected from R³, —OH, ═O, and OR′, wherein R′ is H or —COR″, and R″ is C₁₋₁₀alkyl;
wherein R³ is a substituent comprising an —OH group;
wherein R⁴ and R⁵ are each independently selected from a hydrocarbyl group, H, OH or ═O, or represent a bond with an adjacent atom on the ring of the cyclic compound;
wherein R⁶ and R⁷ are each independently selected from H, OH or ═O, or represent a bond with an adjacent atom on the ring of the cyclic compound.

23. The method of claim 21 wherein said cyclic compound is of formula V, 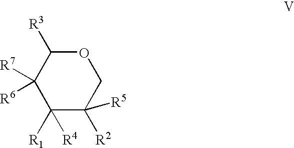
or a derivative thereof, wherein R¹, R², R³, R⁴, R⁵, R⁶ and R⁷ are as defined in claim 17.

24. The composition of claim 22, wherein said cyclic compound is of formula V, 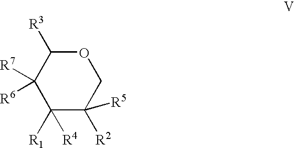
or a derivative thereof, wherein R¹, R², R³, R⁴, R⁵, R⁶ and R⁷ are as defined in claim 17.

25. The method of claim 1, wherein the derivative of the compound of formula I is an ester.

26. The composition of claim 6, wherein the derivative of the compound of formula I is an ester.

27. The method according to claim 25, wherein the ester is formed from an —OH substituent on the cyclic compound, and wherein said ester is of the formula —(CH₂)ₙ—OC(O)—(CH₂)ₚCH₃, wherein n and p are each independently from 1 to 24.

28. The composition according to claim 26, wherein the ester is formed from an —OH substituent on the cyclic compound, and wherein said ester is of the formula —(CH₂)ₙ—OC(O)—(CH₂)ₚCH₃, wherein n and p are each independently from 1 to 24.

29. The method of claim 21, wherein said cyclic compound is selected from one or more of the following:

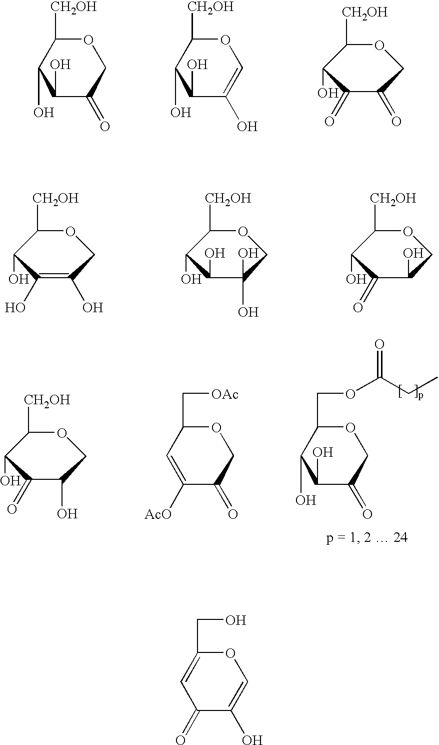

30. The composition of claim 22, wherein said cyclic compound is selected from one or more of the following:

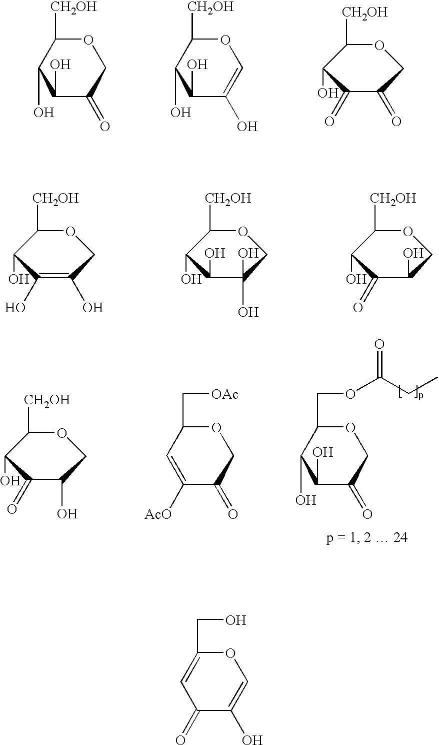

31. The method of claim 1, wherein R³ is or comprises a CH₂OH group.

32. The composition of claim 6, wherein R³ is or comprises a CH₂OH group.

33. The method of claim 1, wherein the cyclic compound comprises a five or a six membered ring.

34. The composition of claim 6, wherein the cyclic compound comprises a five or a six membered ring.

35. The method of claim 1, wherein said compound of formula I is used in combination with one or more of an antioxidant, a preservative and/or a chelator.

36. The composition of claim 6, wherein said compound of formula I is used in combination with one or more of an antioxidant, a preservative and/or a chelator.

37. A pharmaceutical composition comprising
(i) a compound a Formula I, 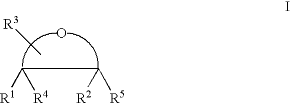
or a derivative thereof,
wherein R¹ and R² are independently selected from —OH, ═O, and OR′, wherein R′ is H or —COR″, and R″ is C₁₋₁₀alkyl;
wherein R³ is a substituent comprising an —OH group;
wherein R⁴ and R⁵ are each independently selected from a hydrocarbyl group, H, OH or ═O, or represent a bond with an adjacent atom on the ring of the cyclic compound; and
(ii) a pharmaceutically acceptable diluent, excipient or carrier, or a mixture thereof.
